(19) United States
(12) Patent Application Publication
(10) Pub. No.: US 2025/0235629 A1
(43) Pub. Date: Jul. 24, 2025

(54) Autoinjector for Automatic Injection of a Product into an Injection Site

(71) Applicant: Becton Dickinson France, Le Pont-de-Claix (FR)

(72) Inventors: Franck Carrel, Saint Jean de Vaulx (FR); Anne Guirimand, Sassenage (FR)

(21) Appl. No.: 18/855,938

(22) PCT Filed: Apr. 5, 2023
(86) PCT No.: PCT/EP2023/059046

§ 371 Date: Oct. 10, 2024

(30) Foreign Application Priority Data
Apr. 11, 2022 (EP) 22305521.1

(51) Int. Cl. A61M 5/32; A61M 5/315

(52) U.S. Cl. CPC A61M 5/326; A61M 5/31501; A61M 5/3204

(57) ABSTRACT

The autoinjector includes a housing extending along longitudinal axis A and configured to receive a medical container having a barrel defining a reservoir for containing a medical product, said barrel having a distal end provided with an injection needle and an opened proximal end configured to receive a plunger rod for pushing a stopper arranged inside the barrel. A needle cover is coupled to and axially movable with respect to said housing between a first extended position, a retracted position, and a second extended position. A cam is rotationally movable around the longitudinal axis A with respect to said housing between a blocking position and a release position, rotation of said cam from the blocking to the release position being caused by the needle cover moving from the first extended position to the retracted position. A plunger rod axially movable inside the housing between an initial position and an injection end position distally located relative to said initial position, under the action of biasing means configured for biasing the plunger rod in a distal direction towards the injection end position. A retainer is arranged for maintaining the plunger rod in the initial position against the action of the biasing means, the retainer being resiliently deformable between a rest position in which the retainer axially abuts against the plunger rod for blocking the plunger rod in said initial position, and a deformed position in which the retainer radially deflect to allow the plunger rod to move in the distal direction. A lock ring is arranged inside the housing for locking the retainer in the rest position. The lock ring is rotatable around the longitudinal axis A between a locking position, in which the lock ring prevents deformation of the retainer, and an unlocking position, in which the lock ring allows for deformation of the retainer, rotation of the lock ring from the locking to the unlocking position being caused by the cam moving from the blocking position to the release position.

### CROSS-REFERENCE TO RELATED APPLICATIONS

This application is the United States national phase of International Patent Application No. PCT/EP2023/059046 filed Apr. 5, 2023, and claims priority to European Patent Application No. 22305521.1 filed Apr. 11, 2022, the disclosures of which are hereby incorporated by reference in their entireties.

### BACKGROUND OF THE INVENTION

### Field of the Invention

The present invention relates to an autoinjector.

### Description of Related Art

In this application, the distal end of a component or of a device is to be understood as meaning the end furthest from the user's hand and the proximal end is to be understood as meaning the end closest to the user's hand. Likewise, in this application, the “distal direction” is to be understood as meaning the direction away from the user's hand, and the “proximal direction” is to be understood as meaning the direction toward the user's hand.

Automatic injection devices are designed for automatic injection of a medical product into an injection site. Autoinjectors usually comprise a housing for receiving a medical container. The medical container has a barrel defining a reservoir for containing the medical product, the barrel having a distal end provided with an injection needle and an opened proximal end receiving a plunger rod for pushing a stopper. The opened proximal end is usually provided with a flange.

Autoinjectors also include a safety shield mechanism moving from an extended to a retracted position to respectively shield or unveil the needle and an injection mechanism for automatically injecting the medical into an injection site. The injection mechanism usually includes a plunger rod for pushing a stopper inside the barrel of the medical container, and an initially compressed spring for moving the plunger in the distal direction. Locking means are provided for maintaining the plunger rod in an initial position in which the plunger rod is axially blocked despite the action of the compressed spring. A release member is typically arranged to release the plunger from the locking means and allow the spring to push the plunger rod in the distal direction to perform injection. A predetermined displacement of the safety shield towards the retracted position is required to allow the release member to unlock the locking means and release the plunger rod.

The document EP2921191 discloses a device for automatic injection of a medical product into an injection site. The device includes a two-part assembly having an upper sub-assembly assembled to a lower sub-assembly. The lower sub-assembly receives a syringe provided with an injection needle. The injection needle is covered by a rigid needle shield. The device further includes a needle cover movable relative to the housing between an extended and a retracted position. The plunger rod is coupled to a compressed spring and maintained in an initial position by a deflectable retainer of an inner cylinder. A push button is arranged at a proximal end of the device for releasing the plunger rod by deflecting the retainer outwardly. Activation of the push button only becomes possible when the needle cover has moved a predetermined distance in the proximal direction towards the retracted position. Besides, activation of the push button requires the end user to press the push button. That is, the end user must perform a specific additional operation to activate the device. Moreover, this operation may not be so easy since the end user must in the same time exert a distal force on the push button and hold the autoinjector pressed against his/her skin.

The document WO2016034407 discloses a signal delaying assembly for a medicament delivery device. The document US2013317432 discloses an autoinjector. The document WO2021094047 discloses a medicament delivery device.

### SUMMARY OF THE INVENTION

There is therefore a need for an autoinjector allowing activation of the injection mechanism without requiring an additional operation from the end user.

Besides, in some prior art autoinjectors, the retainer that retain the plunger rod may creep over time under the stress of the compressed spring. Thus, there is also a need for an autoinjector that is less sensitive to material creep over long storage periods.

Furthermore, to assess the robustness of the autoinjectors, autoinjectors are subjected to drop tests as required in ISO11608. These drop tests usually consist in dropping the autoinjectors at least once from a height of 1 m onto a horizontal floor. There are three drop directions: a drop ‘cap upward’, a drop ‘cap downward’, and a drop with the autoinjector being horizontal. In the first case (drop ‘cap upward’), the push button of the autoinjector hits the floor first. Accordingly, the push button is pressed upon impact on the floor. This may lead to inadvertent activation of the autoinjector.

There is therefore a need for an autoinjector having an improved drop test resistance.

An aspect of the invention is an autoinjector, for automatic injection of a product into an injection site, said autoinjector comprising:
a housing extending along longitudinal axis A and configured to receive a medical container having a barrel defining a reservoir for containing a medical product, said barrel having a distal end provided with an injection needle and an opened proximal end configured to receive a plunger rod for pushing a stopper arranged inside the barrel,
a needle cover coupled to and axially movable with respect to said housing between a first extended position, in which the needle cover at least partially shields the injection needle, a retracted position, in which the needle cover moves proximally with respect to the housing, and a second extended position in which the needle cover moves back in the distal direction to shield the injection needle,
a cam rotationally movable around the longitudinal axis A with respect to said housing between a blocking position and a release position, rotation of said cam from the blocking to the release position being caused by the needle cover moving from the first extended position to the retracted position,
a plunger rod axially movable inside the housing between an initial position and an injection end position distally located relative to said initial position, the plunger rod being configured to push the stopper arranged inside the barrel to expel the medical product,
biasing means for biasing the plunger rod in a distal direction towards the injection end position,
a retainer for maintaining the plunger rod in the initial position against the action of the biasing means, the retainer being resiliently deformable between a rest position in which the retainer axially abuts against the plunger rod for blocking the plunger rod in said initial position, and a deformed position in which the retainer radially deflect to allow the plunger rod to move in the distal direction,
a lock ring arranged inside the housing for locking the retainer in the rest position, said lock ring being rotatable around the longitudinal axis A between a locking position, in which the lock ring prevents deformation of the retainer, and an unlocking position, in which the lock ring allows for deformation of the retainer, rotation of the lock ring from the locking to the unlocking position being caused by the cam moving from the blocking position to the release position.

The autoinjector of the invention allows easier activation of the injection mechanism. Indeed, there is no need to manually press a push button, since the push button has been replaced by a rotative lock ring which automatically moves to an unlocking position when the needle cover moves to the retracted position. The end user has no additional operation to perform, the lock ring rotates automatically. Therefore, the triggering of the injection mechanism including the biasing means and the plunger rod is automatically achieved without need for an additional manual action from the end user.

Besides, the autoinjector provides a better resistance to the drop test ‘cap upward’ because the push button, which was pressed in the axial direction (i.e. the drop direction), has been replaced by the lock ring, which is axially blocked and which can only rotate around the longitudinal axis A. The rotative lock ring being less sensitive to an impact ‘cap upward’, the risks of inadvertently triggering the injection are reduced. It is also contemplated that the lock ring is arranged inside the housing, instead of being arranged at the proximal end thereof (as would be a push button). This arrangement further contributes to making the autoinjector less sensitive to a drop ‘cap upward’.

Eventually, the lock ring blocks outward deflection of the retainer so that the retainer cannot creep over time because of the force exerted by the biasing member on the plunger rod. The autoinjector of the invention is thus less sensitive to material creep over long storage periods.

The retainer preferably includes two diametrically opposite blocking legs provided with a proximal abutment surface for abutting against the plunger rod. Thus, the proximal abutment surface stops distal movement of the plunger rod. The proximal abutment surface may be defined at a proximal side of a radial hook. Preferably, the radial hook is inwardly protruding from a distal end of the blocking legs. The blocking legs axially extend inside the housing. They may be part of an inner sleeve distally protruding from a proximal end of the housing. The retainer may thus be part of the housing and fixed with regard to the housing.

The needle cover, the cam and the lock ring may be separate components.

The biasing means preferably include an injection spring which is compressed in the initial position of the plunger.

The housing is preferably made of a top body and a bottom body assembled to each other by any appropriate securing means, such as snap-fitting means.

The autoinjector of the invention may further include some or all of the features below.

In an embodiment, the lock ring has an axial groove configured to accommodate the retainer when the retainer moves to the deformed position.

In an embodiment, the axial groove includes at least one circumferential stop configured to prevent the retainer from moving back to the rest position when the retainer is engaged in the axial groove.

In an embodiment, the lock ring extends around the retainer. The axial groove may be an inner axial groove.

In an embodiment, the autoinjector includes an axial holder for blocking the lock ring in the axial direction. The autoinjector may include clipping means for allowing clipping of the lock ring to the top body or said axial holder.

In an embodiment, the lock ring includes a side surface configured to abut against the cam when the cam rotates from the blocking position to the release position.

In an embodiment, the side surface is arranged on a drive element engaged with a complementarily shaped drive element of the cam. The drive element may be an outwardly radially extending tab. The tab may be arranged on an axial rib, for instance at a proximal end thereof. The axial rib protrudes from an outer surface of the lock ring and may include a stiffening chamfer.

Possibly, the drive element of the lock ring includes an opposite side surface configured to abut against the cam when the rotates in the opposite direction.

In an embodiment, the drive element of the cam is a notch arranged at the proximal end of the cam. The notch defines a proximal opening for receiving the drive element of the lock ring.

In an embodiment, the lock ring is remote from a proximal end of the housing.

In an embodiment, the lock ring is arranged at a distal end of an inner sleeve axially protruding from the proximal end of the housing.

In an embodiment, the housing has a lateral wall and a transversal wall closing the proximal end of the housing, said transversal wall being integral with the lateral wall.

In an embodiment, the inner sleeve is integral with the transversal wall of the housing.

Possibly, the proximal end of the housing includes a proximally protruding circumferential rim.

In an embodiment, the plunger rod has a distal abutment surface configured to abut against the retainer in the initial position, said distal abutment surface being inclined with regard to the longitudinal axis A. That is, the distal abutment surface is not orthogonal to the longitudinal axis A, and tapers in the distal direction. The plunger rod is thus configured to outwardly deflect the retainer.

Possibly, the retainer includes a proximal abutment surface configured to abut against the plunger rod in the initial position, said proximal abutment surface being inclined with regard to the longitudinal axis A. That is, the proximal abutment surface is not orthogonal to the longitudinal axis A, and tapers in the proximal direction.

Possibly, the plunger rod and the retainer may therefore establish a line contact or conical contact against each other.

In an embodiment, the biasing means include an injection spring arranged around the plunger rod.

Possibly, the plunger rod is symmetrical around the longitudinal axis A.

In an embodiment, the biasing means include an injection spring arranged inside the plunger rod.

### BRIEF DESCRIPTION OF THE DRAWINGS

The terms Fig., Figs., Figure, and Figures are used interchangeably in the specification to refer to the corresponding figures in the drawings.

The invention and the advantages arising therefrom will clearly emerge from the detailed description that is given below with reference to the appended drawings as follows:

FIG. 1 is an exploded view of an autoinjector according to an embodiment of the invention,

FIGS. 2A and 2B are cross-section views of an autoinjector according to an embodiment of the invention,

FIG. 2C is a perspective view of the plunger rod of the autoinjector shown in FIGS. 2A and 2B,

FIGS. 3A and 3B are, respectively, a bottom perspective view and a top perspective view of a lock ring of an autoinjector according to an embodiment of the invention,

FIGS. 4A and 4B are cross-section views of the top body of an autoinjector according to an embodiment of the invention,

FIGS. 5A, 5B and 5C are, respectively, a longitudinal cross-section view, a perspective view and a transversal cross-section view of an autoinjector according to an embodiment of the invention, the autoinjector being idle,

FIGS. 6A, 6B and 6C are, respectively, a longitudinal cross-section view, a perspective view and a transversal cross-section view of an autoinjector according to an embodiment of the invention, the autoinjector being activated,

FIGS. 7A and 7B are cross-section views of an autoinjector according to an embodiment of the invention,

FIGS. 8A and 8B are, respectively, a cross-section view and a perspective view of the plunger rod of the autoinjector shown in FIGS. 7A and 7B,

FIGS. 9A, 9B and 9C are, respectively, a longitudinal cross-section view, a perspective view and a transversal cross-section view of an autoinjector according to an embodiment of the invention, the autoinjector being idle,

FIGS. 10A, 10B and 10C are, respectively, a longitudinal cross-section view, a perspective view and a transversal cross-section view of an autoinjector according to an embodiment of the invention, the autoinjector being activated,

FIGS. 11A and 11B are cross-section views of an autoinjector according to an embodiment of the invention,

FIG. 12 is a cross-section view of an autoinjector according to an embodiment of the invention.

### DESCRIPTION OF THE INVENTION

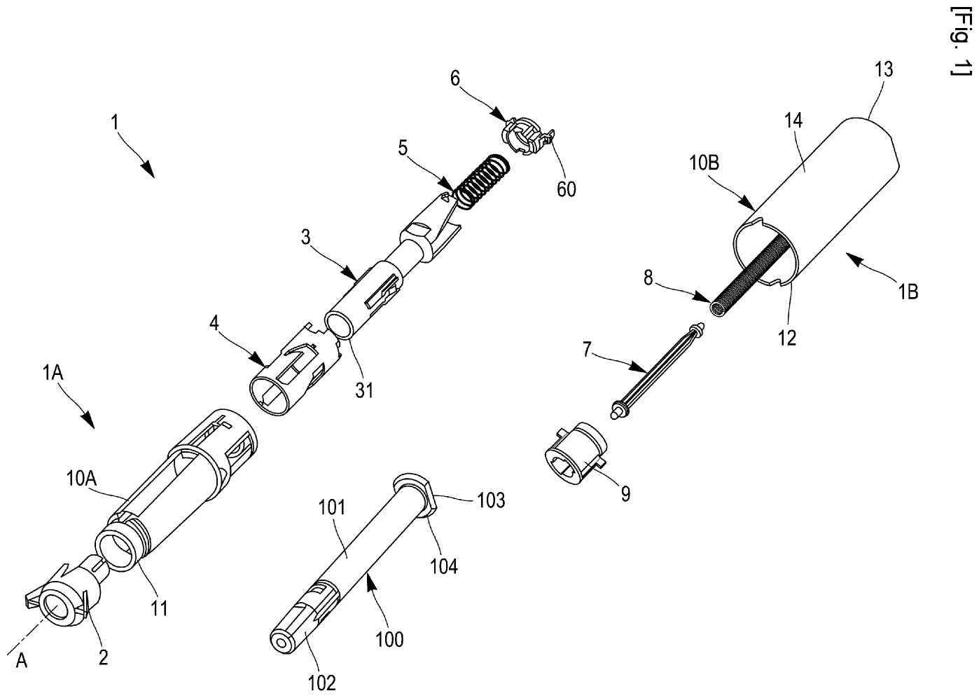

With reference to FIG. 1 is shown an autoinjector 1 according to an embodiment of the invention. The autoinjector 1 is designed for automatic injection of a product into an injection site. The autoinjector 1 extends along a longitudinal axis A. The autoinjector 1 includes a lower sub-assembly 1A and a top sub-assembly 1B assembled to each other by any appropriate securing means such as, for instance, snap-fitting means.

The lower sub-assembly 1A includes a bottom body 10A for receiving a medical container 100, a cap 2 removably attached to a distal end 11 of the bottom body 10A and configured for removing a needle shield 102, a needle cover 3 axially movable along the longitudinal axis A with respect to the bottom body 10A between a first extended position (pre-use position) in which the needle cover 3 at least partially or completely shields an injection needle, a retracted position (injection position) proximally located relative to said first extended position, in which the needle cover 3 moves inside the autoinjector 1 to allow triggering of the injection, and a second extended position (safety position) in which the needle cover 3 moves back in the distal direction so as to safely shield the injection needle. The lower sub-assembly 1A further includes a cam 4 pivotally mounted around the needle cover 3 and the longitudinal axis A, a safety spring 5 for urging the needle cover 3 in the distal direction towards the safety position, and a non-rotatable abutment ring 6 configured to be fixed to the medical container 100 for blocking axial movement of the medical container 100 in the distal direction before activation of the autoinjector 1.

The medical container 100 may be a syringe, for instance a prefilled syringe, and has a tubular barrel 101 defining a reservoir for containing a medical product to be injected. The barrel 101 has a distal end provided with an injection needle (not shown) and a needle shield 102 removably attached to said distal end for protecting and sealing the injection needle. Opposite said distal end, the barrel 101 has an opened proximal end 103 provided with a flange 104 and configured to receive a plunger rod 7.

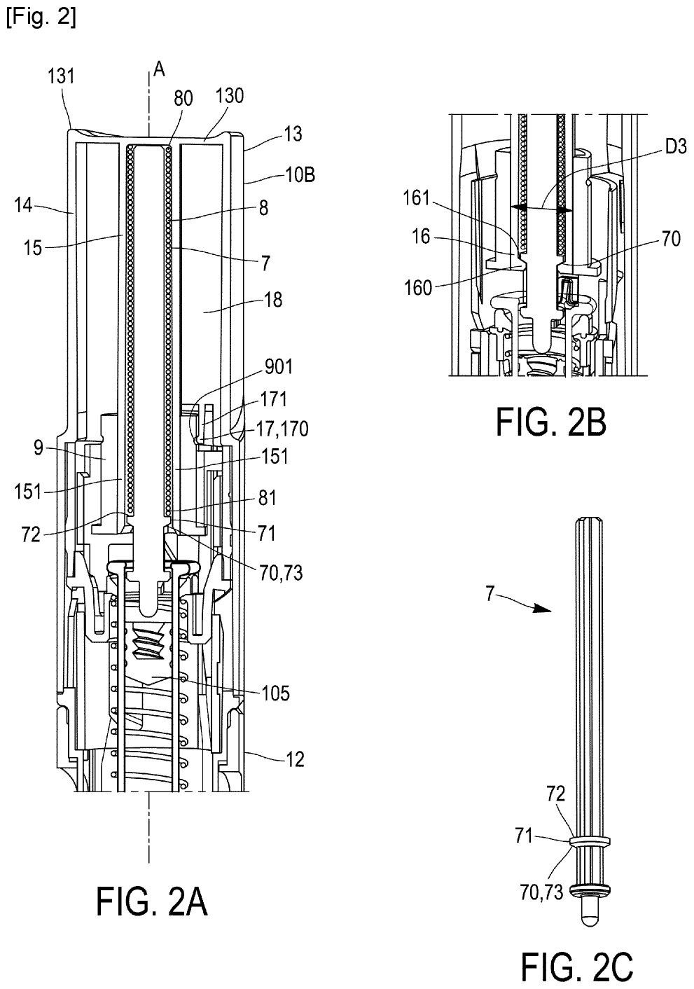

The top sub-assembly 1B includes a top body 10B, a plunger rod 7 axially movable with respect to the top body 10B between an initial position, in which the plunger rod 7 may be away from a stopper 105 (see FIG. 2A) arranged inside the barrel 101, and an injection end position distally located relative to said initial position, in which the stopper 105 abuts against a proximal end of the reservoir defined by the barrel 101. The plunger rod 7 is configured to distally push the stopper 105 for expelling the medical product from the medical container 100. The top sub-assembly 1B also includes biasing means, such as an injection spring 8, for urging the plunger rod 7 in the distal direction and, as illustrated in FIGS. 2A and 2B, a lock ring 9 coupled to the top body 10B and rotatable around the longitudinal axis A. The top body 10B and the bottom body 10A form the housing 10 of the autoinjector 1.

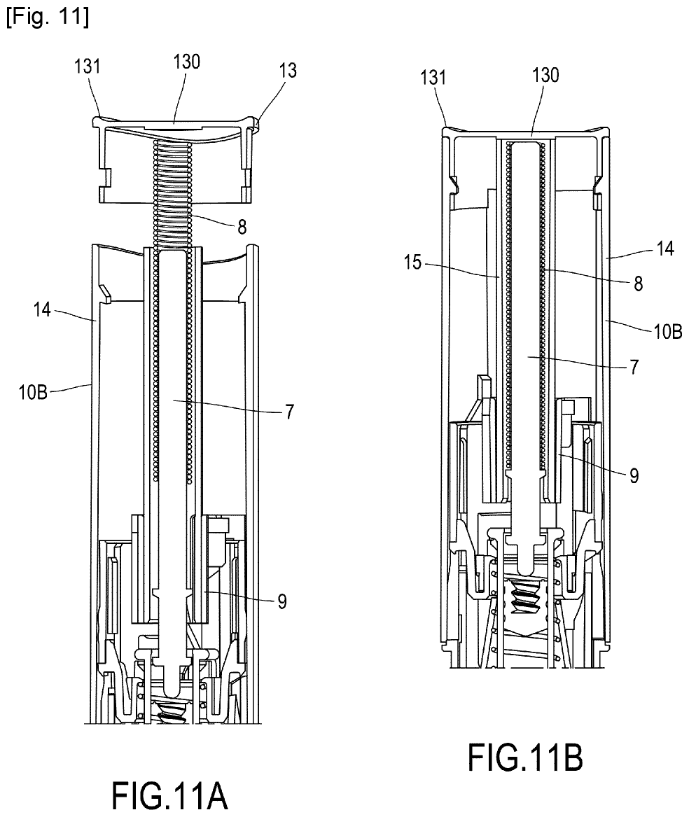

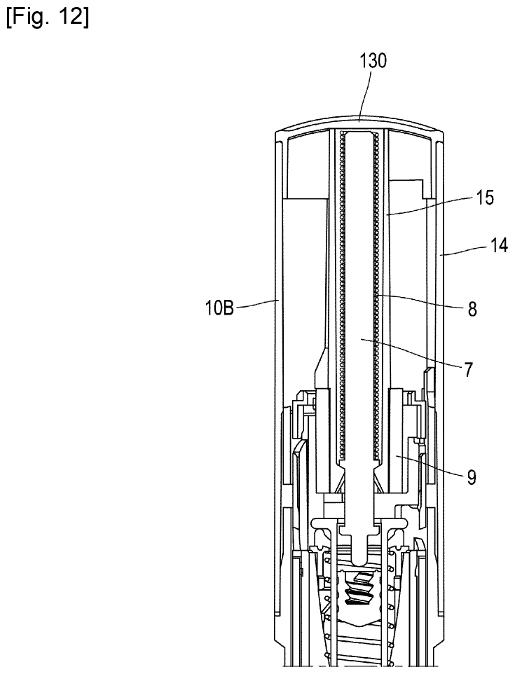

With reference to FIGS. 2A-2B, the top body 10B has a distal end 12 for attachment to the bottom body 10A and a closed proximal end 13. The closed proximal end 13 includes a transversal wall 130 for closing the top body 10B. The transversal wall 130 may extend orthogonal to the longitudinal axis A. Preferably, the transversal wall is integral with a tubular lateral wall 14 of the top body 10B such that the top body 10B is made of a single piece. Therefore, the autoinjector 1 better resists the impact during a drop test ‘cap upward’ (i.e. proximal end of the top body 10B hitting the floor first). In an alternative embodiment (see FIGS. 11A-11B and 12), the transversal wall 130 may be separate from the top body 101B, such that the transversal wall 130 and the top body 10B are two distinct components secured to each other by any appropriate means, such as for instance snap-fitting means, friction-fit or gluing. The closed proximal end 13 may further include a proximally protruding circumferential rim 131 for receiving the user's thumb and thus improve the ergonomics.

With reference to FIGS. 4A-4B, the top body 10B also comprises an inner tubular sleeve 15 defining an inner cavity 150 for accommodating the injection spring 8 and the plunger rod 7 in the initial position, i.e. before activation of the autoinjector 1. The inner sleeve 15 is also configured for guiding the injection spring 8 or the plunger rod 7 from the initial to the injection end position along the longitudinal axis A. The inner sleeve 15 distally protrudes from the closed transversal wall 130 and extends along the longitudinal axis A. With reference to FIGS. 2B, 4A, The inner sleeve 15 includes a retainer 16 arranged at a distal end of the inner sleeve 15 for retaining the plunger rod 7 in the initial position and the injection spring 8 in a compressed state before activation of the autoinjector 1. The retainer may comprise a pair of two diametrically opposite and resiliently deformable blocking legs 16 whose distal end is provided with a radial hook 160 defining a proximal abutment surface 161. The undeformed (FIGS. 4A-4B) blocking legs 16 enables to block distal movement of the plunger rod 7: the plunger rod 7 in the initial position (and more specifically a distal abutment surface 70 of the plunger rod 7) abuts against the radial hook 160, and more specifically against the proximal abutment surface 161 of the blocking legs 16. The deformed blocking legs 16 (not shown) outwardly deflect to move the hooks 160 aside and thus allow distal movement of the plunger rod 7 towards the injection end position. It should be noted that the proximal abutment surface 161 may be inclined with regard to the longitudinal axis A so as to form a ramp configured to favor outward deformation of the blocking legs 16 under the action of the compressed spring 8 after activation of the autoinjector.

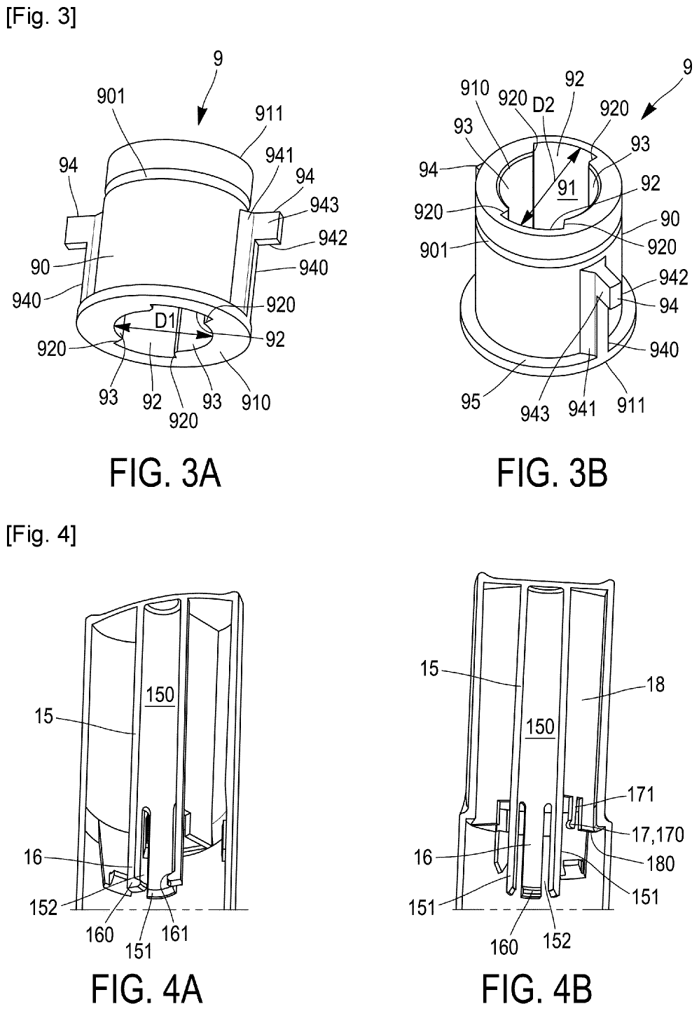

The inner sleeve 15 may further includes bearing means for supporting and guiding rotation of the lock ring 9 around the inner sleeve 15. The bearing means may comprise two diametrically opposite bearing legs 151, which may each extend between the deformable blocking legs 16. The bearing legs 151 and the blocking legs 16 may be regularly distributed around the longitudinal axis A and separated from each other by axial slots 152. They may have the same length. However, the bearing legs 151 are devoid any radial hook and thus do not hamper axial movement of the plunger rod 7. The top body 10B or the inner sleeve 15 may further comprise one or more, for example two diametrically opposite, axial holders 17. As visible in FIG. 4B, the axial holders 17 may include a radial protrusion 170 for engaging a circumferential groove 901 (see FIG. 2A or 3B) of the lock ring 9. The axial holders 17 may be arranged at a distal end of a deformable clipping leg 171, which may protrude from an axially extending spacer plate 18 connecting the lateral wall 14 of the top body 10B and the inner sleeve 15 and whose distal end may serve as a cam axial stop 180. The axial holders 17, the clipping legs 171 and the circumferential groove 901 form clipping means for axially maintaining the lock ring 9 and allowing rotation of the lock ring 9 with respect to the inner sleeve 15. Although not illustrated, the clipping means may alternatively include any other kind of axial hooks, extending for instance from the top body 10B, and configured to engage a complementarily shaped recess or a distal abutment surface arranged for instance on the lock ring 9. In an alternative embodiment (not shown), the axial holders 17 may be formed by an outward radial protrusion at a distal end of the bearing legs 151, the lock ring 9 being in this case assembled to the top body 10B before insertion of the plunger rod 7 and the injection spring 8 inside the inner cavity 150.

It is to be understood that the number of blocking legs 16, respectively bearing legs 151, may vary. For instance, the retainer, respectively, the bearing means, may comprise less or more than two blocking legs 16, respectively bearing legs 151.

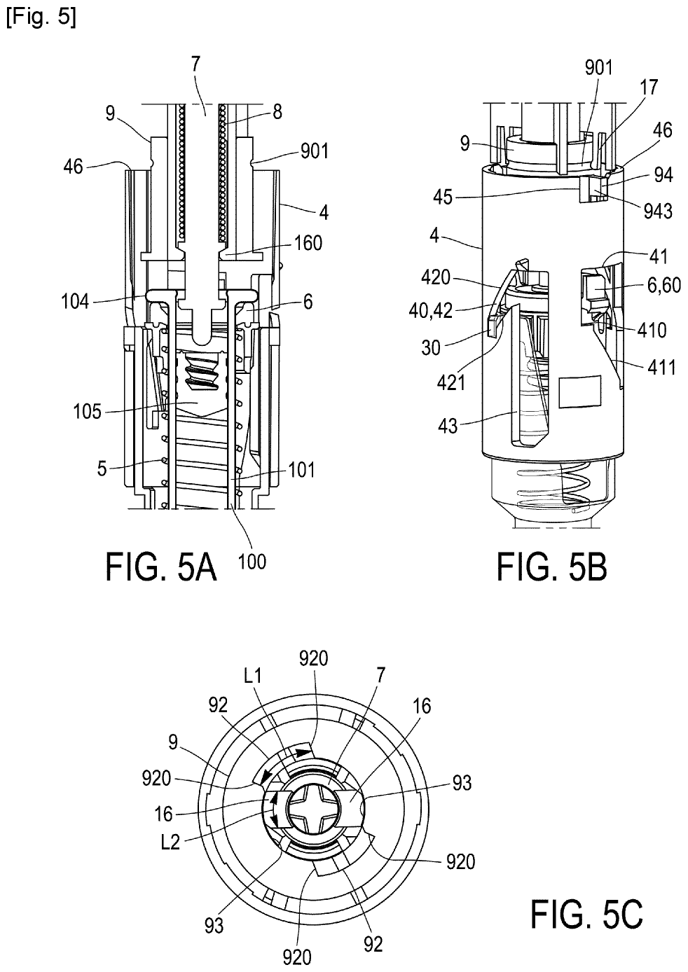

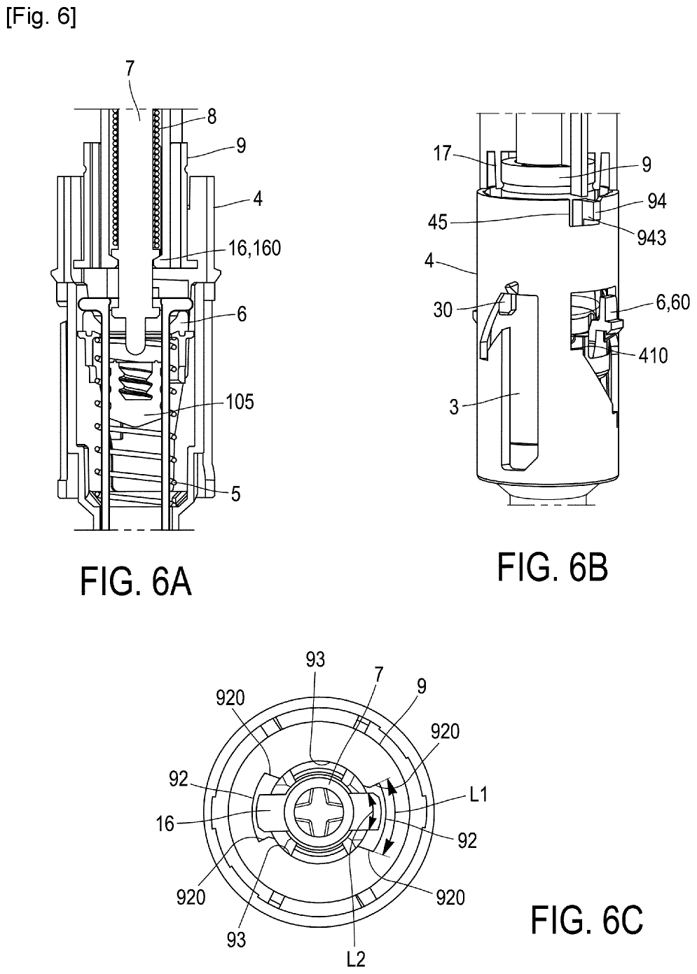

The lock ring 9 is rotatable around the longitudinal axis A and around the inner sleeve 15 between a locking position (FIGS. 2A, 2B, 5A-5C) in which the lock ring 9 maintains the plunger rod 7 in an initial position and the injection spring 8 in a compressed state, and an unlocking position (FIGS. 6A-6C) in which the lock ring 9 allows distal movement of the plunger rod 7 under the action of the injection spring 8. More specifically, the lock ring 9, in its locking position, prevents the blocking legs 16 from outwardly deflecting and, in its unlocking position, allows deformation of the blocking legs 16 such that the plunger rod 7 is released. In the locking position of the lock ring 9, the autoinjector 1 is idle. In the unlocking position of the lock ring 9, the autoinjector 1 is activated.

As visible in FIGS. 2A-2B, the lock ring 9 is arranged at the distal end of the inner sleeve 15, i.e. remote from the proximal end 13 of the top body 10B. Thus, the autoinjector 1 is less sensitive to a drop test ‘cap upward’.

With reference to FIGS. 3A-3B, the lock ring 9 has a cylindrical wall 90 defining an axial cavity 91 for receiving the blocking legs 16 and the bearing legs 151 of the inner sleeve 15. The axial cavity 91 has an opened distal end 910 and an opened proximal end 911 for allowing mounting of the lock ring 9 around the inner sleeve 15. A circumferential groove 901 may be arranged at an outer surface of the cylindrical wall 90 for receiving the axial holder 17 of the top body 10B or of the inner sleeve 15. The axial cavity 91 has two diametrically opposite axial grooves 92 configured to receive the blocking legs 16 when the blocking legs 16 are in their deformed position. The axial grooves 92 are separated by two diametrically opposite blocking lateral walls 93 configured to prevent the blocking legs 16 from outwardly deflecting. Thus, the blocking lateral walls 93 of the lock ring 9 contribute to maintain the plunger rod 7 in the initial position. The blocking lateral walls 93 define a diameter D1 lower than a diameter D2 defined between the axial grooves 92. The diameter D1 may be equal to or slightly greater than an outer diameter D3 (see FIG. 2B) defined by the blocking legs 16 in their rest position. It is contemplated that the lock ring 9 may be symmetrical around the longitudinal axis A.

With reference to FIG. 5C, the autoinjector 1 is idle. The lock ring 9 is in the locking position. The blocking legs 16 of the inner sleeve 15 abut against the blocking lateral walls 93 of the lock ring 9. Thus, the blocking legs 16 cannot deform. The plunger rod 7 is blocked in its initial position. With reference to FIG. 6C, the autoinjector 1 is activated. The lock ring 9 has rotated from the locking to the unlocking position. The angle between the locking and the unlocking position may be comprised between [10°-40° ]. The rotation has been guided by the bearing legs 151 sliding against the blocking lateral walls 93. In this unlocking position, the blocking legs 16 of the inner sleeve 15 now face the axial grooves 92 of the lock ring 9. The blocking legs 16 are no longer prevented from deforming. The distal force exerted by the injection spring 8 on the plunger rod 7 causes the plunger rod 7 to outwardly deflect the blocking legs. The outwardly deflected blocking legs 16 are received in the axial grooves 92 of the lock ring 9. The hook of the blocking legs 16 no longer retain the plunger rod 7 which is released and moves in the distal direction under the action of the injection spring 8.

It is contemplated that the lock ring 9 may include two pairs of circumferential stops 920. The circumferential stops 920 extend at both sides of the axial grooves 92. The circumferential stops 920 are configured to abut against the blocking legs 16 so as to prevent the blocking legs 16 from going back to their rest position against the blocking lateral walls 93 of the lock ring 9 should the lock ring 9 further rotates in the clockwise or counterclockwise direction. The circumferential stops 920 may thus have a surface orthogonal to a circumferential direction. The circumferential length L1 of the axial grooves 92, i.e. the distance between two opposite circumferential stops 920, is preferably greater than the circumferential length L2 of the blocking legs 16.

With reference to FIGS. 3A-3B, the lock ring 9 includes a drive element 94, which may comprise one or two diametrically opposite axial ribs 940 radially protruding from the outer surface of the cylindrical wall 90 for engaging the cam 4. The axial ribs 940 include a tab 94 arranged at a proximal end for engaging the cam 4. A distal end of the axial ribs 940 may join a radial flange 95 surrounding the distal end of the lock ring 9. A stiffening chamfer 941 may run alongside the axial ribs 940. The drive element, more specifically the tab 94, defines a side surface 942 for abutment against the cam 4. Rotation of the lock ring 9 from the locking position to the unlocking position is caused by the cam 4 abutting against this side surface 942, i.e. against the tab 94 of the lock ring 9 while the cam 4 is rotating from a blocking position to a release position. The tab 94 may define an opposite side surface 943 for abutting against the cam 4 such that rotation of the cam 4 in the opposite direction, i.e. back towards the blocking position, causes rotation of the lock ring 9 back towards the locking position.

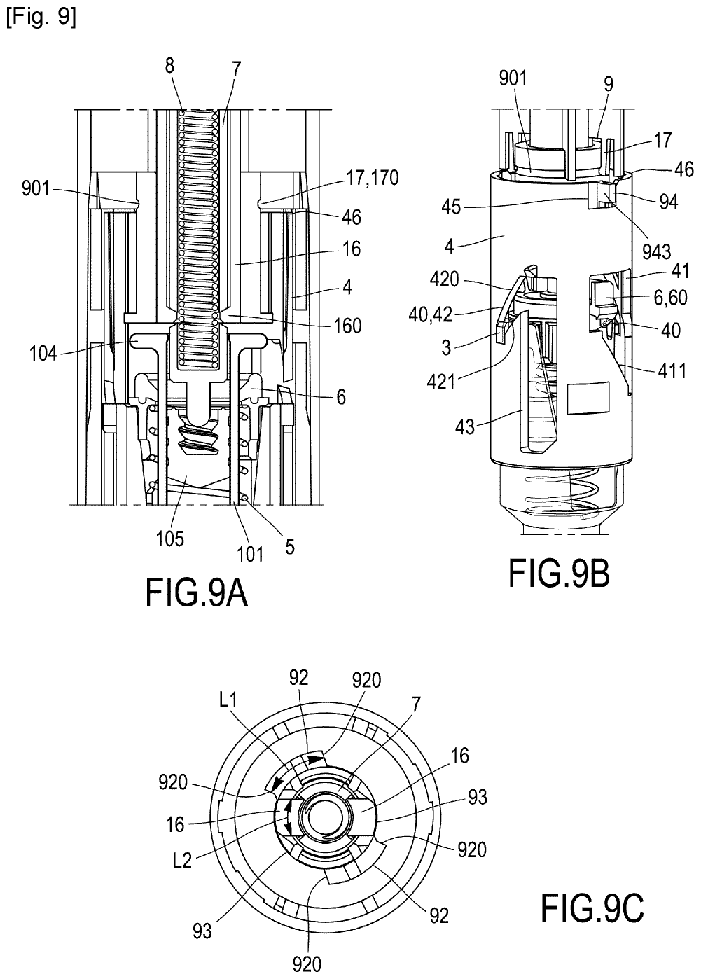

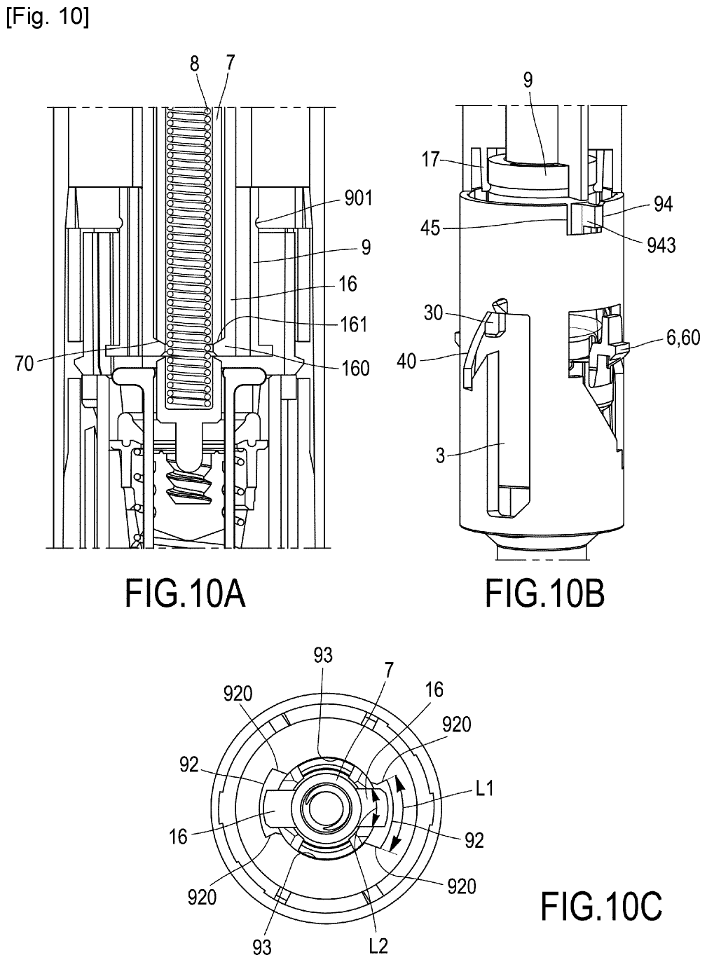

With reference to FIG. 5B, the tubular cam 4 is coupled to the bottom body 10A and is rotatable between a blocking position (FIGS. 5B, 9B), in which the cam 4 is configured to block distal movement of the medical container 100 (and more specifically to block distal movement of the abutment ring 6 fixed to the medical container 100) and a release position (FIGS. 6B, 10B), in which the cam 4 allows for distal movement of the medical container 100. The cam 4 includes a first slot 40 engaged by the needle cover 3 (more specifically a radial lug 30 of the needle cover 3) and a second slot 41 engaged by the abutment ring 6 (more specifically an outwardly extending leg 60 of the abutment ring 6). The abutment of the abutment ring 6 against a distal end of the second slot 41 stops the distal movement of the medical container and thus stops penetration of the injection needle into the injection site. The length of said second slot 41 accordingly defines the injection depth. The first slot 40 has an oblique portion 42 and an axial portion 43 connected to each other at their proximal end 43. The second slot 41 includes a proximal shoulder 410 and a ramp 411 inclined with respect to the longitudinal axis A. In the blocking position, the lug 30 of the needle cover 3 is arranged at a distal end of the oblique portion 42 of the first slot 40, and the abutment ring 6 axially faces the proximal shoulder 410 of the second slot 41. Thus, any distal movement of the medical container 100 before activation of the autoinjector 1 is blocked by the abutment ring 6 abutting against the proximal shoulder 410 of the second slot 41 of the cam 4. As visible in FIG. 5B or 9B, the cam 4 includes a drive element, such as a notch 45, engaging the drive element of the lock ring 9. The notch 45 is thus configured for accommodating the tab 94 of the lock ring 9. Although not shown in the Figures, the notch 45 and the tab 94 may be complementarily-shaped such that rotation of the cam 4 in the clockwise or counterclockwise direction immediately entails rotation of the lock ring 9 in the same direction. The notch 45 may be arranged at the proximal end 46 of the cam 4, and may be proximally opened, to allow insertion of the tab 94 during assembly of the lower sub-assembly 1A and the top sub-assembly 1B of the autoinjector 1.

With reference to the embodiment of FIGS. 2A-2C, the injection spring 8 may be arranged around the plunger rod 7. That is, the plunger rod 7 extends inside the injection spring 8. Since the diameter of the injection spring 8 is greater than the diameter of the plunger rod 7, the injection spring 8 may exert a higher distal force. This allows for instance injection of medical products having a higher viscosity. The injection spring 8 has a proximal end 80 abutting against the transversal wall 130 of the top body 10B and an opposite distal end 81 abutting against an outward flange 71 of the plunger rod 7. More specifically, the injection spring 8 exerts a distal force on a proximal side 72 of the outward flange 71. The outward flange 71 of the plunger rod 7 is configured to abut against the hooks 160 of the blocking legs 16 in the initial position of the plunger rod 7. The opposite distal side 73 of the outward flange 71 may be inclined with regard to the longitudinal axis A. Therefore, the outward flange 71 is configured to deflect the blocking legs 16 due to the action of the injection spring 8 when the lock ring 9 is in the unlocking position. The inclination of the distal side 73 of the outward flange 71 may be similar to the inclination of the proximal abutment surface 161 of the hooks 160, thus establishing a conical contact between the plunger rod 7 and the blocking legs 16. In the illustrated embodiment of FIGS. 2A-2C, the plunger rod 7 may have a X cross-section shape. It should be noted that the plunger rod 7 is symmetrical around the longitudinal axis A, thus easing assembly of the autoinjector 1.

The injection spring 8 is transitionable between a compressed state and a stretched state in which the injection spring 8 is less compressed. When the autoinjector 1 is idle, the injection spring 8 is maintained in the compressed state by the plunger rod 7 abutting against the blocking legs 16 and the blocking legs 16 being prevented from outwardly deflecting by the lock ring 9 being in the locking position. When the autoinjector 1 is activated, i.e. when the lock ring 9 has rotated to the unlocking position, the injection spring 8 extends, thereby pushing the plunger rod 7 in the distal direction up to completion of the injection operation. When the injection is complete, the injection spring 8 is in the stretched state.

The operation of the autoinjector 1 according to the embodiment of FIGS. 2A-2C is described below with reference to the FIGS. 5A-6C and 9A-10C.

As visible in FIGS. 5A-5C and 9A-9C, before activation of the autoinjector 1, the cam 4 is in the blocking position, the needle cover 3 is in the first extended position, the plunger rod 7 is in its initial position, the injection spring 8 is in its compressed state, the lock ring 9 is in the locking position and the blocking legs 16 are in their rest position. The plunger rod 7 is thus prevented from moving in the distal direction by the hooks 160 of the blocking legs 16.

The user first withdraws the cap 2 from the lower sub-assembly 1A. This withdrawal entails removal of the needle shield 102. During the cap 2 removal, the abutment ring 6 may abut against the proximal shoulder 410 of the cam 4 to avoid a back and forth movement of the medical container 100.

The user then places the autoinjector 1 against the injection site. The distal end 31 (FIG. 1) of the needle cover 3 comes against the injection site. As the user presses the autoinjector 1 against the injection site, the needle cover 3 moves from the first extended position to the retracted position. This movement of the needle cover 3 in the proximal direction causes the lug 30 of the needle cover 3 to slide against the upper ramp 420 of oblique portion 42 of the cam 4. Therefore, movement of the needle cover 3 towards the retracted position causes the cam 4 to rotate from the blocking position to the release position.

Since the tab 94 of the lock ring 9 is engaged with the notch 45 of the cam 4, rotation of the cam 4 towards the release position makes the cam 4 push the lock ring 9 in a circumferential direction. The lock ring 9 accordingly rotates from the locking position to the unlocking position.

It should be noted that the autoinjector 1 is activated only when the needle cover 3 moves towards the retracted position, i.e. when the lug 30 of the needle cover 3 moves in the oblique portion 42 towards the axial portion 43 of the first slot 40. If the user changes his/her mind and decides to cancel the injection before activation, then the user just moves the autoinjector 1 away from the injection site. As a result, the safety spring 5 will urge the needle cover 3 back towards the first extended position. The lug 30 of the needle cover 3 will slide against the lower ramp 421 of the oblique portion 42 and the cam 4 will thus rotate back towards the blocking position. The cam 4 will push the tab 94 of the lock ring 9 back in the opposite direction and the lock ring 9 will return to the locking position, still keeping the blocking legs 16 from outwardly deflecting. The injection spring 8 will thus not be released and the plunger rod 7 will still be prevented from moving in the distal direction.

If however the user goes on pressing the autoinjector 1 against the injection site until activation, the needle cover 3 goes on moving towards the retracted position, and the cam 4 eventually reaches the release position (FIGS. 6B and 10B). The lock ring 9, rotated by the cam 4, accordingly reaches the unlocking position (FIGS. 6A, 6C and 10A, 10C). At this stage, there is no possibility to step backward: the injection is triggered and cannot be cancelled. Indeed, the blocking legs 16 now face the axial grooves 92 of the lock ring 9. Due to the distal force exerted by the injection spring 8 on the plunger rod 7, and due to the conical contact between the plunger rod 7 and the blocking legs 16, the blocking legs 16 outwardly deflect and penetrate inside the axial grooves 92. The hooks 160 thus move away from the plunger rod 7. The plunger rod 7 moves towards the injection end position under the action of the injection spring 8, i.e. firstly pushes the medical container 100 so that the injection needle reaches the injection site and then pushes the stopper 105 inside the barrel 101 to expel the medical product. The movement of the medical container 100 in the distal direction may cause the abutment ring 6, fixed to the medical container 100, to abut against the ramp 411 of the second slot 41 of the cam 4. This may, or may not, cause an additional rotation of the cam 4 if required. The lock ring 9 may accordingly rotates further, but this has no other effect than moving the blocking legs 16 from one side to the other side of the axial grooves 92. In any case, the circumferential stops 920 prevent the blocking legs 16 from exiting the axial grooves 92. The blocking legs 16 cannot be deflected back towards their rest position.

When the stopper 105 eventually abuts against a distal end of the reservoir defined by the barrel 101, the injection is complete. An indicator, such as a visual, tactile or audible indicator, may inform the user that the injection is complete.

The user then moves the autoinjector 1 away from the injection site. The needle cover 3 slides in the axial portion 43 of the cam 4, moving from the retracted to the second extended (safety) position under the action of the safety spring 5. A locking mechanism may lock the needle in the safety position to prevent needle stick injuries.

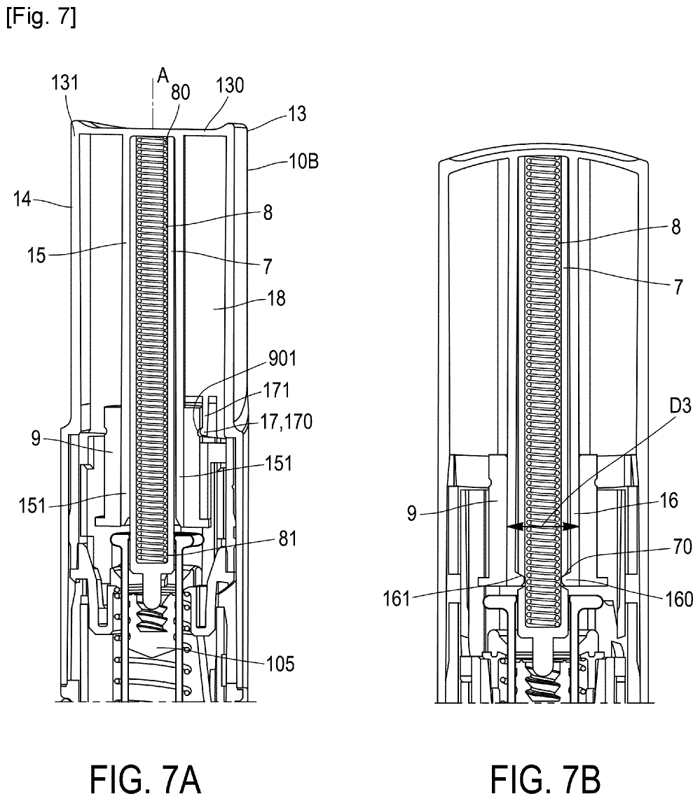

Turning now to FIGS. 7A-10C is shown an autoinjector 1 according to another embodiment of the invention. Similar features with the other embodiments are designated by the same numeral references. The embodiment of FIGS. 7A-10C is similar to the embodiment of FIGS. 2A-6C, apart from the plunger rod 7 and the injection spring 8.

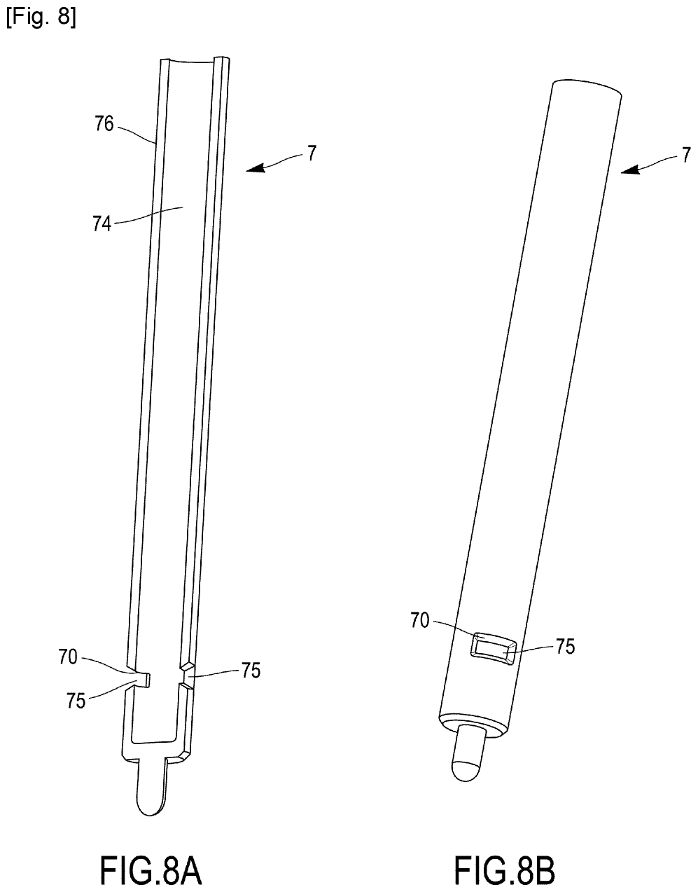

With reference to the embodiment of FIGS. 7A-7B and 8A-8B, the injection spring 8 may be arranged inside the plunger rod 7. That is, the plunger rod 7 extends around the injection spring 8. As visible in FIGS. 7A-7B and 8A-8B, the plunger rod 7 has a tubular wall 76 defining an inner cavity 74 for accommodating the injection spring 8.

Since the diameter of the plunger rod 7 is greater than the diameter of the injection spring 8 and since the injection spring 8 is inside the plunger rod 7, the blocking legs 16 do not abut against the injection spring 8 when the plunger rod 7 moves towards the injection end position. Instead, the blocking legs 16 abut against the tubular wall of the plunger rod 7. This avoids the noise that would be due to the friction between the hooks 160 of the blocking legs 16 and the coils of the injection spring 8. This also makes easier to provide the autoinjector 1 with an indicator, such as a visual, tactile or audible indicator for informing the user that the injection is complete. The injection spring 8 has a proximal end 80 abutting against the transversal wall 130 of the top body 10B and an opposite distal end 81 abutting against a distal end of the inner cavity 74.

The plunger rod 7 has two diametrically opposite windows 75 arranged through the tubular wall 76 for receiving the hooks 160 of the blocking legs 16. The windows 75 have an upper edge configured to abut against the proximal abutment surface 161 of the hooks 160 when the plunger rod 7 is in the initial position. This upper edge corresponds to the distal abutment surface 70 of the plunger rod 7 and may thus be inclined with regard to the longitudinal axis A. Therefore, the upper edge is configured to deflect the blocking legs 16 due to the action of the injection spring 8 when the lock ring 9 is in the unlocking position. The inclination of the upper edge may be similar to the inclination of the proximal abutment surface 161 of the hooks 160, thus establishing a conical contact between the plunger rod 7 and the blocking legs 16.

The operation of the autoinjector 1 according to the embodiment of FIGS. 7A-10C is similar to the one described in connection with FIGS. 2A-6C.

It is readily understandable from the above description that the autoinjector 1 of the invention permits to reduce the steps that are necessary to trigger the injection (the user has no need to push a button and the lock ring 9 automatically rotates when the autoinjector 1 is being pressed against the skin of the user), avoids creep of the blocking legs 16 because of the spring pressure (the lock ring 9 prevents any deformation of the blocking legs 16), and improves the resistance to the drop test ‘cap upward’ (since the injection is longer triggered at the proximal end 13 of the top body 10B).

It is to be understood that the present invention is not limited to the embodiments described above and illustrated in the drawings; rather, the skilled person will recognize that many changes and modifications may be made within the scope of the appended claims.

1. An autoinjector for automatic injection of a product into an injection site, said autoinjector comprising:
a housing extending along longitudinal axis A and configured to receive a medical container having a barrel defining a reservoir for containing a medical product, said barrel having a distal end provided with an injection needle and an opened proximal end configured to receive a plunger rod for pushing a stopper arranged inside the barrel,
a needle cover coupled to and axially movable with respect to said housing between a first extended position, in which the needle cover at least partially shields the injection needle, a retracted position, in which the needle cover moves proximally with respect to the housing, and a second extended position in which the needle cover moves back in the distal direction to shield the injection needle,
a cam rotationally movable around the longitudinal axis A with respect to said housing between a blocking position and a release position, rotation of said cam from the blocking to the release position being caused by the needle cover moving from the first extended position to the retracted position,
a plunger rod axially movable inside the housing between an initial position and an injection end position distally located relative to said initial position, the plunger rod being configured to push the stopper arranged inside the barrel to expel the medical product,
biasing means for biasing the plunger rod in a distal direction towards the injection end position,
a retainer for maintaining the plunger rod in the initial position against the action of the biasing means, the retainer being resiliently deformable between a rest position in which the retainer axially abuts against the plunger rod for blocking the plunger rod in said initial position, and a deformed position in which the retainer radially deflect to allow the plunger rod to move in the distal direction, and
a lock ring arranged inside the housing for locking the retainer in the rest position, said lock ring being rotatable around the longitudinal axis A between a locking position, in which the lock ring prevents deformation of the retainer, and an unlocking position, in which the lock ring allows for deformation of the retainer, rotation of the lock ring from the locking to the unlocking position being caused by the cam moving from the blocking position to the release position.

2. The autoinjector according to claim 1, wherein the lock ring has an axial groove configured to accommodate the retainer when the retainer moves to the deformed position.

3. The autoinjector according to claim 2, wherein the axial groove comprises at least one circumferential stop configured to prevent the retainer (16) from moving back to the rest position when the retainer is engaged in the axial groove.

4. The autoinjector according to claim 1, wherein the lock ring extends around the retainer.

5. The autoinjector according to claim 1, wherein the autoinjector comprises an axial holder for blocking the lock ring in the axial direction.

6. The autoinjector according to claim 1, wherein the lock ring comprises a side surface configured to abut against the cam when the cam rotates from the blocking position to the release position.

7. The autoinjector according to claim 6, wherein the side surface is arranged on a drive element engaged with a complementarily shaped drive element of the cam.

8. The autoinjector according to claim 7, wherein the drive element of the cam is a notch arranged at the proximal end of the cam.

9. The autoinjector according to claim 1, wherein the lock ring is arranged at a distal end of an inner sleeve axially protruding from the proximal end of the housing.

10. The autoinjector according to claim 1, wherein the housing has a lateral wall and a transversal wall closing a proximal end of the housing, said transversal wall being integral with the lateral wall.

11. The autoinjector according to claim 10, wherein the inner sleeve is integral with the transversal wall of the housing.

12. The autoinjector according to claim 1, wherein the plunger rod as a distal abutment surface configured to abut against the retainer in the initial position, said distal abutment surface being inclined with regard to the longitudinal axis A.

13. The autoinjector according to claim 1, wherein the biasing means comprise an injection spring arranged around the plunger rod.

14. The autoinjector according to claim 1, wherein the biasing means comprise an injection spring arranged inside the plunger rod.
